(19) United States
(12) Patent Application Publication
(10) Pub. No.: US 2018/0028373 A1
(43) Pub. Date: Feb. 1, 2018

(54) STORAGE SYSTEM FOR FEMININE HYGIENE PRODUCT AND METHOD FOR USE

(71) Applicant: Rachel June Donovan, New York, NY (US)

(72) Inventor: Rachel June Donovan, New York, NY (US)

(21) Appl. No.: 15/225,265

(22) Filed: Aug. 1, 2016

(51) Int. Cl. A61F 13/551; A61F 13/26; B65D 75/52

(52) U.S. Cl. CPC A61F 13/5519; A61F 13/5514; A61F 13/5515; A61F 13/5518; A61F 13/55185; B65D 75/527; A61F 13/263; A61F 13/266

(57) ABSTRACT

A system including a first storage pocket having an inner cavity with an absorbent pad positioned therein. The system further includes a second storage pocket having an inner cavity with a tampon positioned therein, wherein the second storage pocket is directly coupled to the first storage pocket.

The present invention relates generally to a storage system for feminine hygiene products, wherein the system includes various storage pockets.

### BACKGROUND

Tampons have been known in the art for some time. Most tampons consist of an applicator which houses an absorbent insert and facilitates the deployment into the vaginal canal thereof. Absorbent pad have also been known in the art for some and generally consist of a piece of absorbent material that is placed above an undergarment to absorb any bodily fluids during the menstrual cycle. However, many existing tampon and/or absorbent pad systems do not have sufficiently convenient packaging and/or storage options.

### SUMMARY

In one embodiment the invention is a system for storing an absorbent pad and a tampon, comprising two directly coupled storage pockets wherein a first storage pocket comprises an inner cavity with an absorbent pad positioned therein, and a second storage pocket comprises an inner cavity with a tampon positioned therein.

In another embodiment the invention is a method for using a system for storing feminine hygiene products, comprising two directly coupled storage pockets wherein a first storage pocket comprises an inner cavity with an absorbent pad positioned therein, and a second storage pocket comprises an inner cavity with a tampon positioned therein. The method optionally includes separating storage pockets along the tear guideline. The method optionally includes the additional step of disposing of a feminine hygiene product by positioning it in a storage pocket and closing the storage flap over the mouth of the pocket, thus sealing the feminine hygiene product inside for sanitary disposal.

### BRIEF DESCRIPTION OF THE DRAWINGS

FIG. 1 is a top view of one embodiment of the storage system, shown in a compact configuration;

FIG. 2 is a top view of the storage system of FIG. 1, shown in an expanded configuration with the storage flaps in a closed position;

FIG. 3 is a side perspective view of the system of FIG. 2, with the storage flaps in an open position;

FIG. 4 is a top view of the system of FIG. 3, with a tampon and an absorbent pad partially removed from an associated storage cavity;

FIG. 5 is a top view of the system of FIG. 4 wherein the storage pockets have been separated along a tear guideline and a storage flap has been closed; and

FIG. 6 is a top view of a storage pocket of FIG. 5 for standalone use and/or disposal.

### DETAILED DESCRIPTION

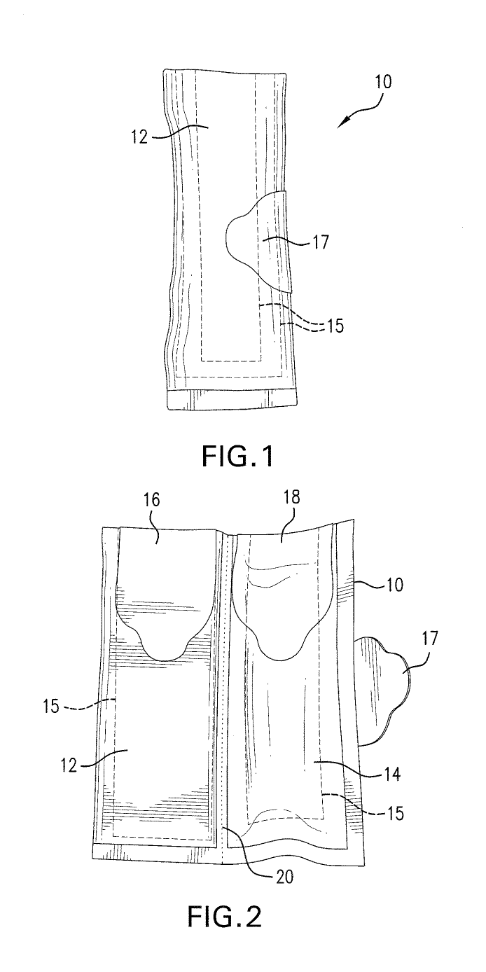

As shown in FIGS. 1-6, in one embodiment the system 10 can include a first pocket 12 and a second pocket 14 coupled together. The first pocket 12 and second pocket 14 each have an associated inner cavity 15, and can each have roughly the same size and shape, such as rectangular in top view in one case. The first pocket 12 and second pocket 14 can be coupled together along a tear guideline 20. The tear guideline 20 can provide an area or line along which the system 10 is predisposed to tear, and may be able to be manually torn with a relatively low tearing force. As will be described in greater detail below, the tear guidelines 20 can also serve as a pivot/hinge line about which each pocket 12, 14 can pivot. In the illustrated embodiment the tear guideline 20 bisects the system 10 longitudinally.

The system 10/pockets 12, 14 can have any of a wide variety of shapes and sizes. In one embodiment, however, each pocket 12, 14 can have a width (extending horizontally in FIGS. 1-6) of about one inch, and a height (extending vertically in FIGS. 1-6) of about four inches. The system 10, and more particularly the pockets 12, 14 can be made of any of a wide variety of materials. For example, the system 10 and pockets 12, 14 can be made of a relatively thin, flexible and pliable polymer such as plastic, or paper, cardboard or combinations thereof, or various other materials.

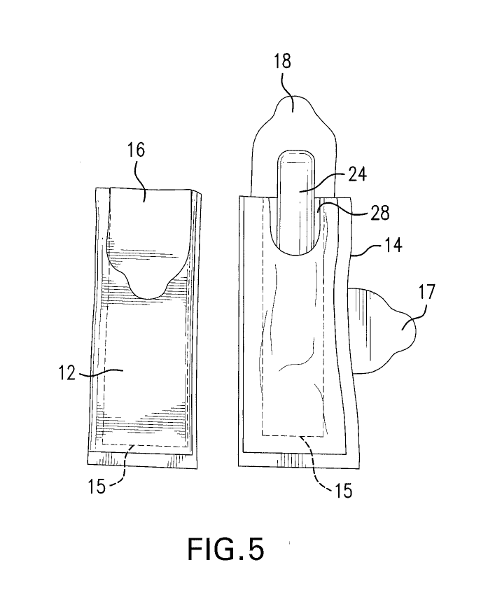

The tear guideline 20 may include or take the form of perforation lines, fold lines, crease lines, areas of weakness, score lines, and as noted above is able to be manually torn to separate the first storage pocket 12 and second storage pocket 14 from each other. FIG. 5 shows the first storage pocket 12 and the second storage pocket 14 after they have been entirely torn along the tear guideline 20 and separated from each other.

Each of the first storage pocket 12 and the second storage pocket 14 has a mouth 26, 28 that opens to an associated inner cavity 15. Each of the first storage pocket 12 and the second storage pocket 14 also has an associated storage flap 16, 18 that controls access to the associated inner cavity 15. The storage flap 16 of the first storage pocket 12 is attached to the upper, shorter side of the first storage pocket 12 and can be configured to selectively cover the mouth 26 of the first storage pocket 12 and/or selectively close the associated inner cavity 15. The storage flap 18 of the second storage pocket 12 is attached to the upper, shorter side of the second storage pocket 14 and can be configured to selectively cover the mouth 28 of the second storage pocket 14 and/or selectively close the associated inner cavity. As show in FIG. 2, by folding the storage flaps 16, 18 over the respective mouths 26, 28, access to the inner cavities 15 of each of the storage pockets 12, 14 is blocked.

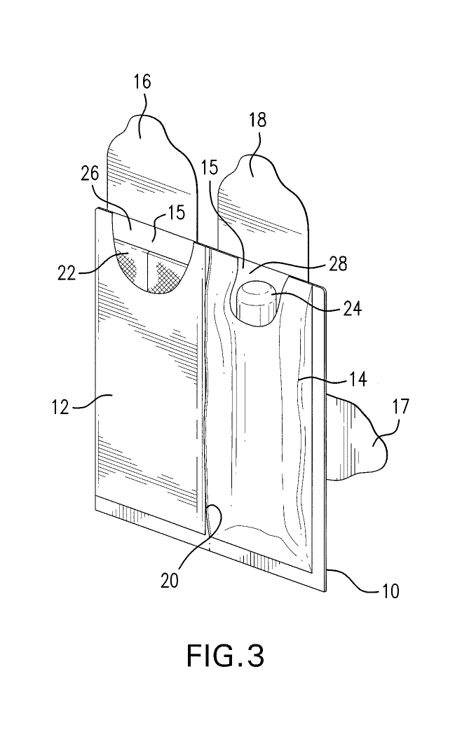

The storage flaps 16, 18 can each be moved to an open position as shown in FIG. 3, allowing access to the inner cavities 15 of each of the storage pockets 12, 14 and any feminine hygiene products contained therein. In FIG. 3, an absorbent pad 22 is positioned in the inner cavity 15 of the first pocket 12, and a tampon 24 is positioned in the inner cavity 15 of the second pocket 14. Of course, the position of the tampon 24 and absorbent pad 22 can be reversed. The storage flaps 16, 18 shown are shown in FIG. 3 in an open position, thus allowing access to the inner cavities 15 of each storage pocket 12, 14. In one embodiment, the system 10 includes structure or means configured to releasably secure the storage flaps 16, 18 to the associated storage pockets 12, 14 when in their closed positions, such as a releasable adhesive or a hook and loop fastening material such as VELCRO® hook and loop fastening material.

FIGS. 2-4 illustrate the system 10 in an expanded configuration wherein the first storage pocket 12 and the second storage pocket 14 are unfolded and positioned side by side. The system 10 can also be positioned in a compact configuration, wherein the first storage pocket 12 and the second storage pocket 14 are folded along the tear guideline 20 such that the pockets 12, 14 are generally aligned, as shown in FIG. 1.

The system 10 may include a retaining flap 17 attached to a longitudinal side of the one of the storage pockets 12, 14 (the second storage pocket 14 in the illustrated embodiment). In the illustrated embodiment the retaining flap 17 is attached to longitudinal edge of the second storage pocket 14, and extends generally perpendicular to the edge where the retaining flap 17 is attached. In one embodiment, the retaining flap 17 includes structure or means (such as a releasable adhesive or a hook and loop fastening material) configured to releasably secure the distal end of the retaining flap 17 to the pocket 12 to maintain the system 10 in its compact position.

The system 10 can store a tampon 24 and an absorbent pad 22 (collectively termed “feminine hygiene products”). The tampon 24 can include a generally cylindrical applicator which houses an absorbent insert and facilitates deployment of the absorbent insert into the vaginal canal thereof. Such applicators are typically formed of two coaxial, telescoping pieces, with an inner piece acting as a plunger to force the absorbent insert through the top of an outer piece during deployment. The absorbent insert of the tampon 24 can be formed from cotton, rayon, or a cotton-rayon mixture and can vary in thickness. Individual tampons are typically stored in packages that are separate from other feminine hygiene products, and in packages that are not resealable.

The absorbent pad 22 can consist of a rectangular-shaped piece of absorbent material that is placed above a wearer's undergarment to absorb any bodily fluids during the menstrual cycle or otherwise. The absorbent pad 22 can be formed from cotton, rayon, or a cotton-rayon mixture and can vary in thickness. Some absorbent pads 22, also called “maxi-pads” can absorb regular menstrual flow without the assistance of an additional feminine hygiene product such as a tampon 24. Other absorbent pads 22 are much thinner and are typically known as panty-liners. The absorbent pad 22 disclosed and used in the system 10 can include or take the form of a panty-liner, a maxi-pad, a sanitary napkin, a sanitary pad, and the like. Individual absorbent pads 22 are typically stored in packages that are separate from other feminine hygiene products, and in packages that are not resealable.

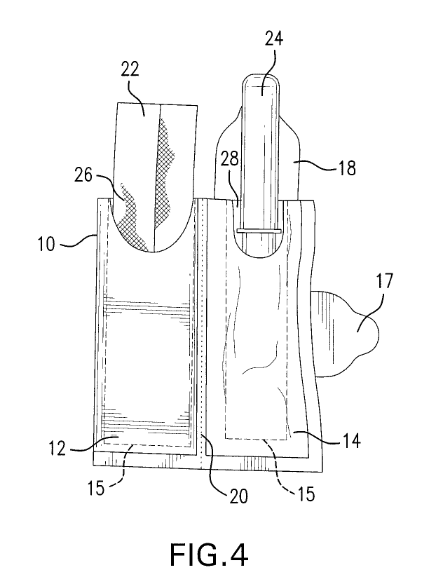

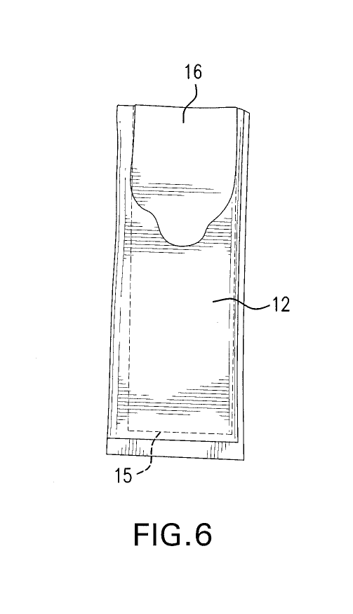

As outlined above the system 10 packages together an absorbent pad 22 and a tampon 24 for convenient use. The system 10 can be boxed, shipped and stored in its compact configuration, as show in FIG. 1. In addition, a user may store the system 10 in its compact configuration shown in FIG. 1, for example in a purse or the like. The system 10 can be retained in its closed position by the retaining flap 17 as outlined above. When the user desires to use a tampon 24 and/or absorbent pad 22, the user can release the retaining flap 17, and pivot/move the system 10 to the expanded configuration, as shown in FIG. 2. Next, as shown in FIG. 3, the user can pivot open one or both of the storage flaps 16, 18 to access one or both of the tampon 24 or absorbent pad 22, which can then be removed (FIG. 4) from its associated inner cavity 15 and used as desired. If only one of the tampon 24 or absorbent pad 22 is desired to be used, the user can tear the system 10 along the tear guideline 20 (FIG. 5). In this manner, the empty storage pocket 12, 14 can be discarded, while the storage pocket 12, 14 that is not empty can be retained (i.e. replaced in user's purse) (FIG. 6) for storage or later use.

Sometimes, due to the nature and heaviness of a menstrual cycle, the user will require the protection of both a tampon 24 and an absorbent pad 22. Thus the system 10 ensures both items 22, 24 are immediately available. In other cases, a user may not be sure whether the user will need either a tampon 24 or an absorbent pad 22. Thus the system 10 ensures that both are available.

The system 10 can also be used as a sanitary means of disposing the tampon 24 or absorbent pad 22 after use. A user could place the used tampon 24 or absorbent pad 22 inside the empty inner cavity 15 of one of the storage pockets 12, 14. The user can fold the storage flap 16, 18 over the mouth 26, 28 of the associated inner cavity 15 to the mouth 26, 28. The storage flap 16, 18 can be affixed to the outer surface of the pocket 12, 14, thus sealing the tampon 24 or absorbent pad 22 inside. Such system allows for sanitary disposal of the tampon 24 or absorbent pad 22 into an appropriate trash receptacle.

It will be understood that the foregoing is only illustrative of the principles described herein, and that various modifications can be made by those skill in the art without departing form the scope and spirit of the invention. Moreover, features of embodiments described herein may be combined and/or rearranged to create new embodiments.

What is claimed is:

1. A system comprising:
a first storage pocket having an inner cavity with an absorbent pad positioned therein; and
a second storage pocket having an inner cavity with a tampon positioned therein, wherein the second storage pocket is directly coupled to the first storage pocket.

2. The system of claim 1 wherein the first storage pocket is pivotally coupled to the second storage pocket.

3. The system of claim 1 wherein the first storage pocket is pivotally coupled to the second storage pocket along a tear guideline.

4. The system of claim 3 wherein the system is movable between a compact configuration, wherein the first storage pocket and the second storage pocket are folded along the tear guideline such that the pockets are generally aligned, and an expanded configuration wherein the first storage pocket and the second storage pocket are unfolded and positioned side by side.

5. The system of claim 4 further including a retaining flap permanently coupled to one of the first or second pockets and releasably coupleable to the other one of the first or second pockets, wherein the retaining flap is configured to form a releasable connection to maintain the system in the compact configuration.

6. The system of claim 3 wherein the system is tearable along the tear guideline such that the first and second storage pockets are entirely separable from each other.

7. The system of claim 1 wherein the first storage pocket and the second storage pocket are each generally the same size and shape.

8. The system of claim 7 wherein the first storage pocket and the second storage pocket are each generally rectangular in top view.

9. The system of claim 1 wherein each storage pocket has a mouth providing access to the associated inner cavity, and wherein each storage pocket has a storage flap configured to selectively cover the mouth to control access to associated inner cavity.

10. A system comprising:
first storage pocket having an inner cavity with an absorbent pad positioned therein; and
a second storage pocket having an inner cavity with a tampon positioned therein, wherein the second storage pocket is pivotally coupled to the first storage pocket.

11. A method for using a system comprising:
accessing a system including a first storage pocket having an inner cavity with an absorbent pad positioned therein, and a second storage pocket having an inner cavity with a tampon positioned therein; and
pivotally moving the first storage pocket relative to the second storage pocket along a tear guideline.

12. The method of claim 11 further comprising the step of removing the absorbent pad from the inner cavity of the first storage pocket and removing the tampon from the inner cavity of the second storage pocket.

13. The method of claim 11 further comprising folding the system along the tear guideline into a compact configuration wherein the first storage pocket and second storage pocket are generally aligned.

14. The method of claim 11 further comprising the step of separating the first storage pocket and second storage pocket from each other by manually tearing the system along the tear guideline.
